(19) United States
(12) Patent Application Publication
(10) Pub. No.: US 2025/0003999 A1
(43) Pub. Date: Jan. 2, 2025

(54) AUTOMATED SYSTEM AND METHOD FOR ISOLATION AND/OR EXTRACTING SUBSTANCE BY MAGNETIC BEAD

(71) Applicant: TAIWAN ADVANCED NANOTECH INC., Taoyuan City (TW)

(72) Inventors: CHIEN-HSING CHIEN, Taoyuan City (TW); JUNG-HSIEN HUNG, Taoyuan City (TW); KAN-SANG MOK, Taoyuan City (TW); SHENG-YEN HUANG, Taoyuan City (TW)

(73) Assignee: TAIWAN ADVANCED NANOTECH INC., Taoyuan City (TW)

(21) Appl. No.: 18/759,002

(22) Filed: Jun. 28, 2024

Related U.S. Application Data
(60) Provisional application No. 63/524,398, filed on Jun. 30, 2023.

(51) Int. Cl. G01N 35/00; C12N 15/10; G01N 1/34

(52) U.S. Cl. CPC G01N 35/0098; C12N 15/1013; G01N 1/34; G01N 2035/00465

(57) ABSTRACT

The present disclosure provides an automated system and a method for isolation and/or extracting substance by magnetic beads. With the preset program, sample isolation and the following assay such as PCR or Immunoprecipitation may be performed in single instrument of automated system.

### BACKGROUND OF THE INVENTION

### Field of the Invention

The present disclosure relates to an automated system and method for isolating and/or extracting substance extraction. Particularly, the disclosure relates to an automated system and method for cell isolation and substance extraction performing by magnetic beads, wherein all the procedures are integrated in one device.

### Background

Cell isolation plays a very important role in the biotechnology industry. Purification of specific cells can ensure the correctness of subsequent cultures to ensure that there will be no errors in various experiments. Cell isolation may be performed by differential centrifugation, density gradient centrifugation, velocity sedimentation, isopycnic sedimentation, flow cytometer, cell electrophoresis or magnetic beads.

Sample extraction such as nucleoid acid extraction, protein extraction may be performed in different ways, such as centrifugation, solvent method, or magnetic bead method, etc. After the sample is purified, qualitative, quantitative experiments, or other analysis can be proceeded.

However, the above-mentioned various analysis methods need to be carried out separately through their own machines or by manpower, which is time-consuming and labor-intensive. In addition, automated extraction platform is currently a trend, and it is necessary to develop a platform that can simultaneously integrate various analysis methods, such as sample isolation, extraction, and detection. Therefore, there is a need for an automated system that can perform multiple analysis at once.

### SUMMARY OF THE INVENTION

This research and development result is an automated system for isolating samples and/or extracting substance by magnetic beads. This technology can be used in the field of academic research and biotechnology industry, for sample manipulation, molecular diagnosis, cell therapy development, clinical pathogen detection, entry-exit inspection operations, and other nucleic acid analysis-related applications. Moreover, the isolated cells with high viability can be used for subsequent culture. The characteristics of automation process of this technology are suitable for understaffed units. The simple interpretation method reduces the professional threshold required by operators. In addition, the integrated kit and extraction system realize a single device that can complete cell isolation, sample purification, nucleic acid extraction and molecular detection at one time.

For the purpose of the present disclosure, providing an automated system for isolating and/or extracting substance by magnetic beads, comprising: a switchable module, comprising magnetic rotary mixers, the magnetic rotary mixers comprising: a plurality of magnetic rods for generating magnetism, configured to be retractable from the switchable module; a plurality of spin shafts for mounting spin tips, and the plurality of magnetic rods extend therein; and a first motor for moving the plurality of magnetic rods vertically; an auto stage, comprises: a plate holder, which allows a plate place thereon; a second motor for moving the plate horizontally; a third motor for moving the switchable module vertically; and a temperature controlling plate, disposed under the plate holder for controlling the temperature of the plate; a controller for performing a pre-set program of movement; and a shell housing the switchable module and the auto stage.

Preferably, the switchable module comprises a first magnetic rotary mixer and a second magnetic rotary mixer, the first magnetic rotary mixer comprises 4 or 8 channels for extending and/or retracting the magnetic rods and the second magnetic rotary mixer comprises 4 or 8 channels for extending and/or retracting the magnetic rods. The numbers of channels of the magnetic rotary mixer are not limited, the user can adjust the numbers of channels upon the actual applications.

Preferably, the switchable module comprises rotary switchable module or linear switchable module.

Preferably, the system further comprises a fourth motor for switching the first and second magnetic rotary mixers horizontally rotation.

Preferably, the first to fourth motors are step motors.

Preferably, the temperature control plate is a thermoelectric sheet.

Preferably, the shell further comprises a HEPA.

Preferably, the shell further comprises an opening for the auto stage moving in and out.

Preferably, the opening further comprises a door.

Preferably, the system further comprises a detection unit.

Preferably, the detection unit comprises fluorescent test unit, culture test unit or cytotoxicity test unit.

For another purpose of the present disclosure, providing a method for sample isolation and/or extraction by using the automated system, comprising introducing samples, reagents and magnetic beads into the plates; and conducting a sample isolation and/or extraction steps; wherein the sample isolation and/or extraction steps are completed on the automated system at once.

Preferably, the sample comprises cell or cell derivatives.

Preferably, the sample extraction step comprises extracting protein, nucleic acid or cell derivatives.

Preferably, the method further comprises performing an immunoprecipitation assay.

Preferably, the method is performed by a pre-determined program in the automated system.

Preferably, the method further comprises a detection step.

Preferably, the detection step comprises fluorescent test, culture test or cytotoxicity test.

The automated system may allow isolation and the following assays being performed in one single instrument, which can save more time and manpower, and can avoid possible pollution during the transmission between assays.

### Technology Comparison

Cell isolation is a critical technique in biological research and clinical applications, used to separate specific cell types from a heterogeneous population. Two common methods for cell isolation are manual cell isolation and magnetic bead automatic cell isolation. Here's a detailed comparison of the two:
Magnetic Bead Automatic Cell Isolation Vs. Manual Cell Isolation

### Method:

Magnetic bead isolation involves using magnetic nanoparticles coated with antibodies that bind to specific cell surface markers. Once the target cells are labeled, they are separated using a magnetic field.

Manual cell isolation typically involves using flow cytometry (FACS—Fluorescence-Activated Cell Isolation) or microscopy-based techniques where cells are sorted based on their physical and fluorescent characteristics.

### Advantages:

1. Speed: Faster processing of large volumes of cells compared to manual sorting.
2. Ease of Use: Simpler protocol with less technical expertise required.
3. Scalability: Easily scalable for sorting large numbers of cells.
4. Viability: Generally gentler on cells, resulting in higher viability post-sorting.
5. Cost-Effective: Lower initial setup cost and operational expenses compared to flow cytometry.

### Disadvantages:

1. Specificity: Limited to sorting cells with known surface markers that have corresponding magnetic beads.
2. Purity: May have lower purity compared to flow cytometry, especially if non-specific binding occurs.
3. Limited Parameters: Cannot sort based on multiple parameters simultaneously; relies on the presence of specific surface markers.
4. Magnetic Bead Removal: Post-sorting steps may be required to remove magnetic beads from the sorted cells, which can add to the complexity.

| | Manual Cell Isolation | Magnetic Bead Automatic Cell |
|---|---|---|
| Feature | (Flow Cytometry) | Isolation |
| Precision | High | Moderate |
| Speed | Slow | Fast |
| Technical Skill | High | Low |
| Cost | High | Moderate |
| Cell Viability | Moderate | High |
| Purity | High | Moderate |
| Flexibility | High (multiple | Low (single parameter) |
| | parameters) | |
| Scalability | Limited | High |
| Stress on Cells | High | Low |
| Marker | Fluorescent markers | Surface antigens with |
| Requirement | | corresponding magnetic beads |

Advantages of Automated Instruments for Combination of Magnetic Bead Cell Isolation with Other Substance Extraction Steps in the Present Disclosure

Automated instruments that utilize magnetic bead cell separation and direct nucleic acid extraction, or integrate additional substance extraction steps, provide numerous benefits. These systems significantly increase efficiency by streamlining workflows, allowing for the simultaneous processing of multiple samples, thereby reducing manual operations and processing time. Enhanced consistency and reproducibility are achieved as automated systems minimize human errors, ensuring uniform handling of samples and reagents, which leads to more reliable and repeatable results compared to manual methods or separate function execution. The risk of contamination is reduced through decreased manual intervention and limited sample exposure to external environments. Automation also enhances scalability, allowing easy expansion of experiments by combining different reagent kits for various functions. In the present disclosure, the integration of multiple workflow steps into a single instrument (from cell separation to nucleic acid extraction or other functions) reduces the need for multiple devices and simplifies the workflow. These instruments are user-friendly, featuring intuitive interfaces and preset procedures, making them accessible to users with limited technical experience. Overall, by combining various functions into one automated process, these instruments save significant time, enabling researchers to focus on data analysis and interpretation.

These charts provide a concise description and summary of the advantages of automated instruments using magnetic bead cell isolation with other substance extraction steps.

| Advantage | Description |
|---|---|
| Increased | Streamlines workflows, allows simultaneous |
| Efficiency | processing of multiple samples, significantly reduces |
| | manual operations and processing time. |
| Enhanced | Minimizes human errors, ensures consistent sample |
| Consistency and | and reagent handling, and produces reliable and |
| Reproducibility | repeatable results. |
| Reduced | Lowers contamination risk by minimizing manual |
| Contamination | interventions and reducing sample exposure to |
| Risk | external environments. |
| Scalability | Allows easy scaling of experiments, combining |
| | reagent kits to perform various functional experiments. |
| Multi-step | Integrates various workflow steps, reducing the need |
| Integration | for multiple devices and simplifying the workflow. |
| User-Friendly | Features intuitive interfaces and preset procedures, |
| Operation | making it easy for users with limited technical |
| | experience to operate. |
| Time Savings | Combines multiple functions into one automated |
| | process, significantly saving time and allowing |
| | researchers to focus on data analysis and |
| | interpretation. |

| Feature | Automated Instruments |
|---|---|
| Efficiency | High-Combines multiple functions, reduces manual |
| | operations, and processes multiple samples |
| | simultaneously. |
| Consistency | High-Minimizes human errors, ensures reliable and |
| | repeatable results. |
| Contamination | Low-Reduces manual interventions and sample |
| Risk | exposure to external environments. |
| Scalability | High-Easily scales experiments, combines different |
| | reagent kits for various functions. |
| Workflow | Comprehensive-Integrates cell separation, nucleic |
| Integration | acid extraction, and other functions into a single |
| | instrument. |
| Ease of Use | User-friendly-Intuitive interfaces and preset |
| | procedures, suitable for users with limited technical |
| | experience. |
| Time Efficiency | High-Combines functions into one process, allowing |
| | more focus on data analysis and interpretation. |

### BRIEF DESCRIPTION OF THE DRAWINGS

FIG. 1 illustrates a block diagram of the automated system 1 according to an embodiment of the present disclosure.

FIG. 2 illustrates a perspective view of the automated system 1 according to an embodiment of the present disclosure.

FIG. 3 illustrates a perspective view of the automated system 1 from another side according to an embodiment of the present disclosure.

FIG. 4 illustrates a perspective view of setting plates 202 on the plate holder 201 of the automated system 1 according to an embodiment of the present disclosure.

FIG. 5 illustrates a perspective view that the plates 202 is moved under the rotary switchable module 100 of the automated system 1, and a partial enlarged view of the magnetic rod 111 according to an embodiment of the present disclosure.

FIG. 6 illustrates a perspective view of the plate holder 201 holding plates 202 according to an embodiment of the present disclosure.

FIG. 7 illustrates a perspective view from upper and lower angle of the rotary switchable module 100 according to an embodiment of the present disclosure.

FIG. 8 illustrates another embodiment of the automated system 2 of the present disclosure, wherein the switchable module is a linear switchable module 100a.

FIG. 9 illustrates another embodiment of the automated system 2 of the present disclosure, wherein the linear switchable module 100a is moved down toward the plates 202.

FIG. 10 illustrates a perspective view from upper angle of the linear switchable module 100a of automated system 2.

FIG. 11 illustrates a perspective view from lower angle of the linear switchable module 100a of automated system 2.

FIG. 12 illustrates a flow chart of the method for cell isolation, extracting substances and PCR assay according to an embodiment of the present disclosure.

FIG. 13 illustrates a flow chart of the method for cell isolation, extracting substance and Immunoprecipitation assay according to an embodiment of the present disclosure.

FIG. 14 Flowchart of the automatic cell isolation process in automated system 1.

FIG. 15 shows the efficiency of automatic cell isolation according to an embodiment of the present disclosure.

FIG. 16 shows the result of Purity of CD3+ cells automatically isolated by the 4 channels of automated system 1.

FIG. 17 shows the result of Purity of CD3+ cells automatically isolated by the 8 channels of automated system 1.

FIG. 18 shows flowchart of nucleic acid extraction from MB-cells.

FIG. 19 shows the result of the CD45 immunoprecipitation from the total protein lysate of the isolated CD3+ cells by the 4 channels of automated system 2.

### DETAILED DESCRIPTION OF THE EMBODIMENTS

The embodiments are described in detail below with reference to the related drawings. However, these embodiments can be implemented in different forms, but are not the only form of implementing or utilizing the specific embodiments of the disclosure claimed in this application, and therefore should not be construed as a limitation on the above-mentioned embodiments. The features of various specific embodiments as well as method steps and sequences for constructing and operating these specific embodiments are encompassed in the detailed description. However, other embodiments may also be utilized to achieve the same or equivalent function and sequence of steps. Rather, these embodiments are provided so that this specification can be thoroughly and completely disclosed and will fully convey the spirit of the disclosure to people having ordinary skill in the art to which the disclosure pertains. Similar reference numerals in the figures refer to similar elements. In the following description, well-known functions or structures will not be described in detail so as not to repeat unnecessary details in the embodiments.

Unless otherwise defined, all technical phrases and terms used herein have the same meaning as commonly understood by people having ordinary skill in the art to which this disclosure pertains. In case of conflict, the present specification including the definitions shall prevail.

Without conflicting with the context, the singular nouns used in this specification cover the plural form of the noun; and the plural nouns used also cover the singular form of the noun. In addition, in the specification and the claims, expressions such as “at least one” and “one or more” have the same meaning, and both mean that one, two, three or more are included.

Notwithstanding that the numerical ranges and parameters setting forth the broad scope of the disclosure are approximations, the related numerical values in the specific examples have been presented as precisely as possible. However, any numerical value inherently and inevitably contains the standard deviation resulting from individual testing methods. As used herein, “about” generally means that the actual value is within plus or minus 10%, 5%, 1% or 0.5% of a particular value or range. Alternatively, the word “about” means that the actual value lies within an acceptable standard error of the mean, as considered by people having ordinary skill in the art to which this disclosure pertains. Except in the examples, or unless expressly stated otherwise, all ranges, quantities, values and percentages used herein (e.g., to describe material amounts, time periods, temperatures, operating conditions, quantitative ratios, and the like) are understood to be modified by “about”. Therefore, unless otherwise stated to the contrary, the numerical parameters disclosed in this specification and the claims are all approximate numerical values, which can be changed as required. At a minimum, these numerical parameters should be construed to mean the number of significant digits indicated and the numerical values obtained by applying ordinary rounding. Numerical ranges are expressed herein as being from one endpoint to the other endpoint or between the endpoints; and unless otherwise indicated, the numerical ranges recited herein are inclusive of the endpoints.

There are kinds of instruments for isolation, extraction and qualitative or quantitative experiment. However, most of the instruments only have a single function and lack integrated functions. Costumers have to buy various instruments for their individual function and have to transmit the samples between instruments to complete a whole analysis, which increases the risk of pollution and also time-consuming.

Therefore, the present disclosure provides an automated system that not only can perform individual experiment, but also can perform different experiments or assays in one single instrument.

In one embodiment, the automated system 1 for isolating and/or extracting substance by magnetic beads, comprising: a rotary switchable module 100, an auto stage 200, a controller 300 for performing a pre-set program of movement; and a shell 500 housing the rotary switchable module 100 and the auto stage 200 and the rotary switchable module 100 comprises magnetic rotary mixers 110/120. The magnetic rotary mixers 110/120 comprises a plurality of magnetic rods 111, a plurality of spin shafts 102 and a first motor. The plurality of magnetic rods 111 generate magnetism for attracting magnetic beads, which are configured to be retractable from the magnetic rotary mixers 110/120. The magnetic rotary mixers 110/120 may each comprises 4, 6, 8, 12 magnetic rods 111 depends on the needs, which is not limited in the present disclosure. A plurality of spin shafts 102 are configured for mounting spin tips 103, and the plurality of magnetic rods 111 may extend therein. The first motor are configured for moving the plurality of magnetic rods 111 vertically.

In one embodiment, the spin tips 103 may be removed by further extending the magnetic rods 111. The auto stage 200 comprises a plate holder 201, which allows a plate 202 place thereon; a second motor for moving the plate 202 horizontally; a third motor for moving the rotary switchable module 100 vertically; and a temperature controlling plate 210, disposed under the plate holder 201 for controlling the temperature of the plate 202. The number of the plate 202 may be adjust depends on the needs.

In one embodiment, the switchable module comprises rotary switchable module 100 or linear switchable module 100a. The rotary switchable module 100 may comprise a motor for switching the magnetic rotary mixers 110/120 horizontally rotation. The linear switchable module 100a may comprise rotary mixers 110a/120a stacked in a direction along to the rail of the auto stage 200 and the rotary mixers 110a/120a may move along the direction the same. Therefore, comparing to the automated system 1, the width of the shell 500 of the automated system 2 may be reduced.

In one embodiment, the motors mentioned above may be step motors for precise control. The temperature control plate 210 may be a thermoelectric sheet or other temperature controller that can provide precise temperature control. The shell 500 of the automated systems 1 or 2 may comprise opening 500 with door 501 for allowing plates 202 being set in. In one embodiment, the automated systems 1 or 2 may further comprise detection unit, such temperature sensor, fluorescent test unit, culture test unit or cytotoxicity test unit. The detection unit may check the result of the assay or assure the procedure of the assays run in a right condition.

In one embodiment, a method for sample isolation and/or extraction are performed by the automated system, comprising introducing samples, reagents and magnetic beads into the plates; and conducting a sample isolation and/or extraction steps. The sample may comprise cell, cell derivatives or suitable substance that may be combined to magnetic beads. The sample extraction step may comprise extracting protein, nucleic acid or cell derivatives. The isolation and/or extraction steps are completed on the automated system at once with a pre-determined program in the automated system.

In another embodiment, the method may further comprise performing an immunoprecipitation assay or detection steps such as fluorescent test, culture test or cytotoxicity test. The assays or detection steps may also be performed at once with a pre-determined program in the automated system.

Herein after, the automated system 1 of embodiments of the present disclosure will be described in detail corresponding with the drawings.

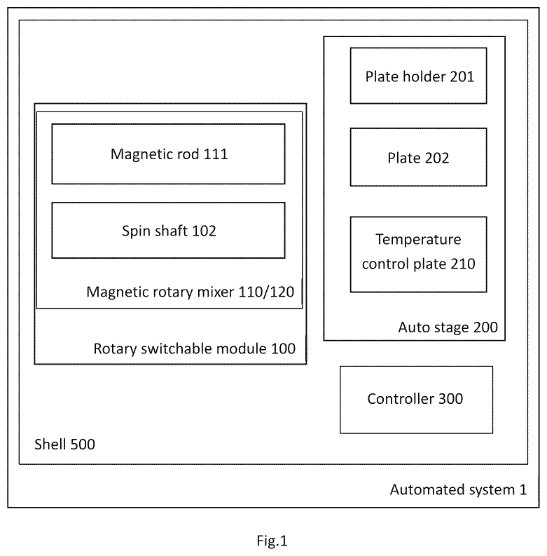

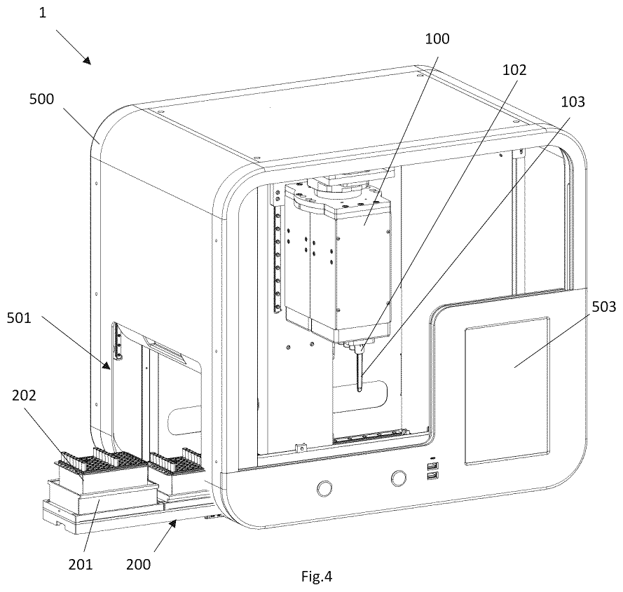

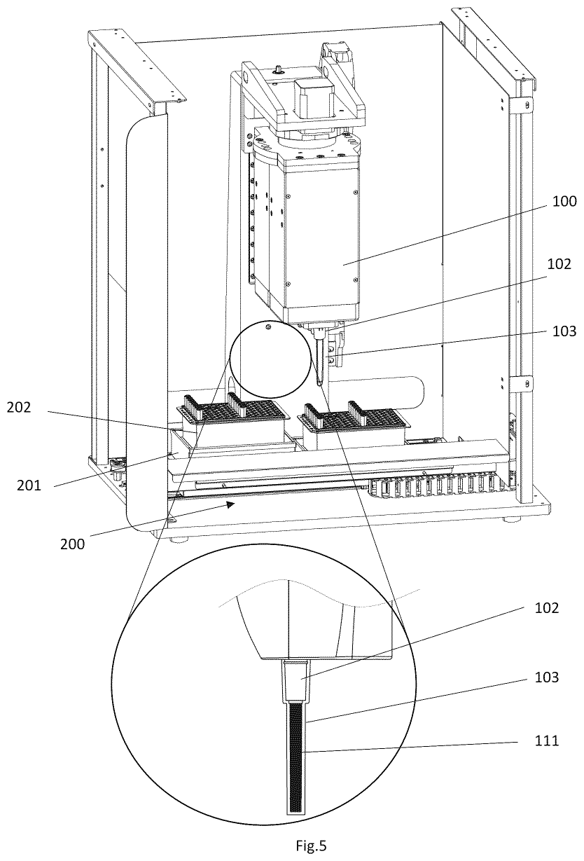

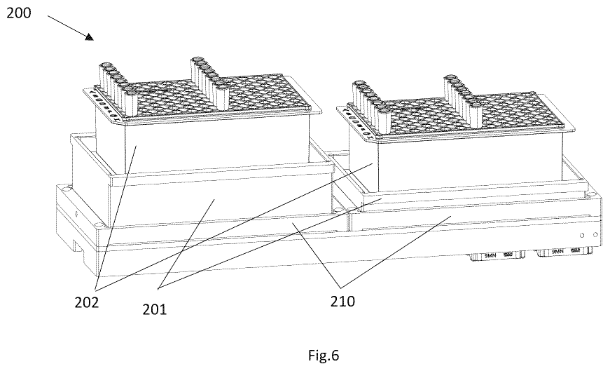

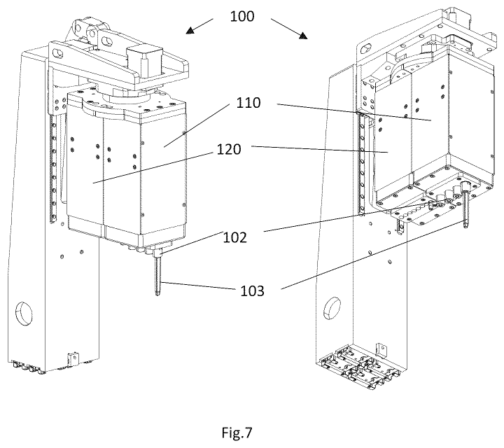

For one embodiment of the present disclosure, as shown in FIGS. 1 to 7, the automated system 1 for isolating and/or extracting substance by magnetic beads comprises a rotary switchable module 100 and an auto stage 200. The magnetic rotary mixer comprises magnetic rotary mixers 110/120 comprising a plurality of magnetic rods 111 for generating magnetism, configured to be retractable from the magnetic rotary mixers 110/120 (see FIGS. 5 and 7). A plurality of spin shaft 102 disposed thereof are for mounting spin tips 103, and the plurality of magnetic rods 111 extend therein. A first motor (not shown) is disposed therein for moving the plurality of magnetic rods 111 vertically. The auto stage 200 comprises a plate holder 201, which allows a plate 202 place thereon; a second motor (not shown) for moving the plate 202 horizontally; a third motor (not shown) for moving the rotary switchable module 100 vertically; a temperature control plate 210 disposed under the plate holder 201 for controlling the temperature of the plate 202; and a controller 300 for performing a pre-set program of movement. A shell 500 is further disposed for housing the rotary switchable module 100 and the auto stage 200. In one embodiment, the first motor, second motor and third motor are step motors to precisely control the movement of the rotary switchable module 100, the magnetic rods 111 therein and the plate holder 201, respectively.

Please refer to FIG. 7, in one embodiment, the rotary switchable module 100 comprises a first magnetic rotary mixer 110 having 4 channels or a second magnetic rotary mixer 120 having 8 channels of magnetic rods 111 to fit with 24 or 96 well plate. In another embodiment, the rotary switchable module 100 comprises first and second magnetic rotary mixer 110 and 120 with magnetic rods 111 to fit with 24 and 96 well plate in one instrument. To perform the switch of between first and second magnetic rotary mixer 110 and 120, the rotary switchable module 100 comprises a fourth motor to switch the first and second magnetic rotary mixer 110/120 horizontally according to the needs of the operator. The fourth motor may be a step motor to control the horizontal rotation of the first and second magnetic rotary mixer 110/120. The number of channels of the magnetic rotary mixer is not limited as described above, any suitable number may be used according to the need of the user. For example, the number of channels of the magnetic rotary mixer may be 4, 6, 8, 10, 12 or any suitable number.

In one embodiment, the temperature control plate 210 can be performed as a cooler or a heater, preferably the temperature control plate 210 is made by a thermoelectric sheet which can cool down and heat up the plate 202 in one element.

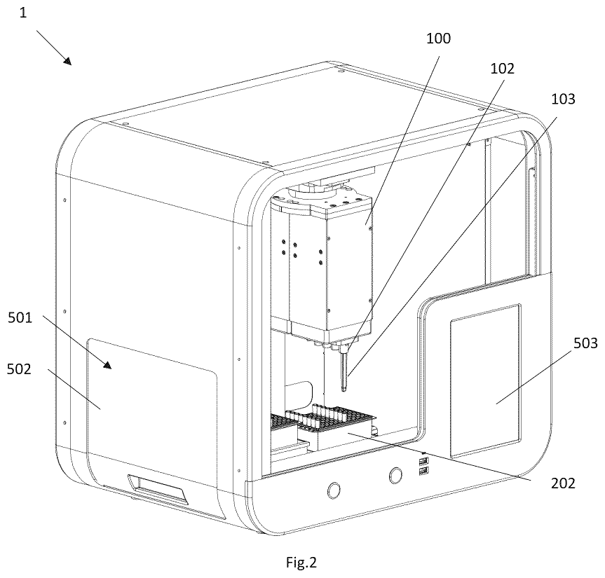

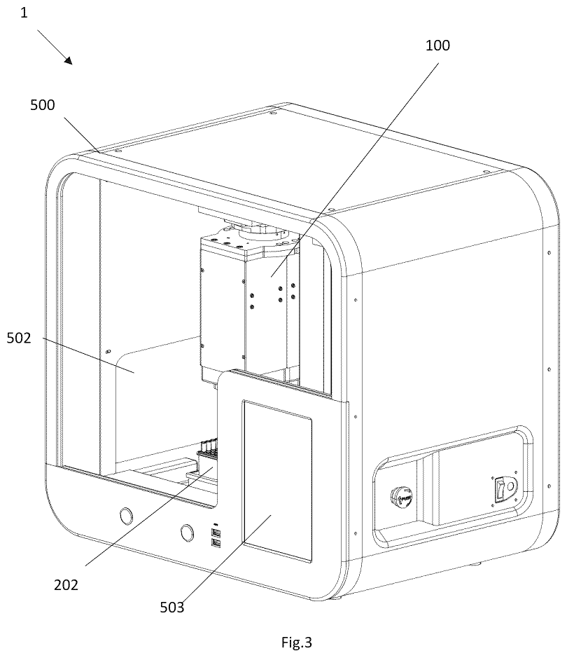

Please refer to FIGS. 2 and 3, in one embodiment, shell 500 may comprise a HEPA disposed therein to avoid possible pollution. In one another embodiment, shell 500 may comprise an opening 501 for disposing the plate 202 on the plate holder 201 of the auto stage 200, the plate holder 201 may be moved inside and outside of the shell 500. The opening 501 may further comprise a door 502 to avoid possible pollution. The door 502 may be a shutter or a cover in any suitable form. A control panel 503 may be further disposed on the shell 500 in any suitable position, preferably on the front side of the shell 500.

With the above-mentioned technical features, the automated system 1 may perform individual assay or perform two or more assay at once.

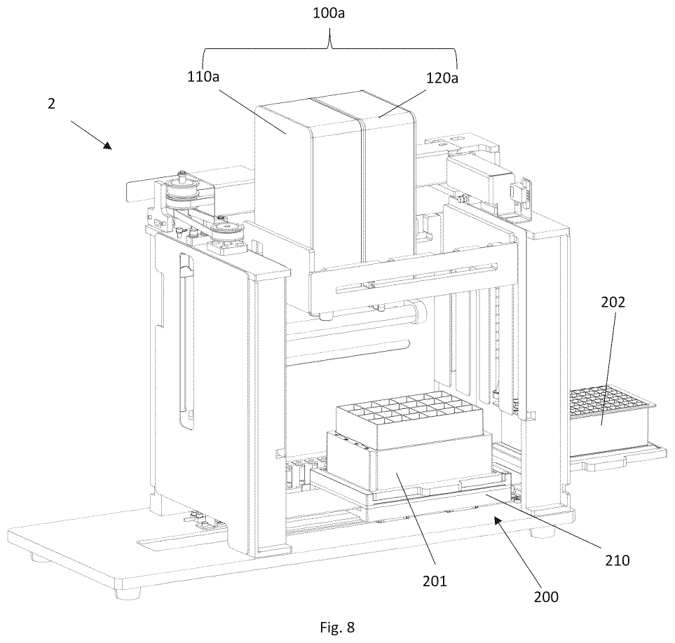

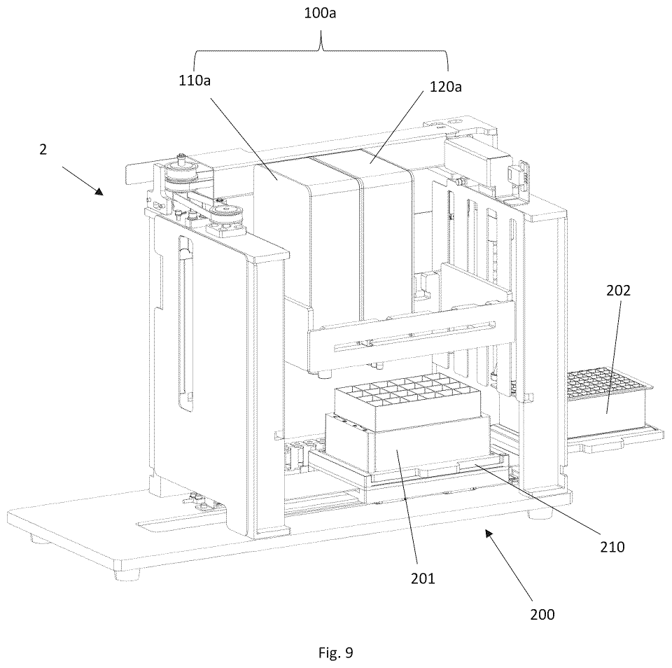

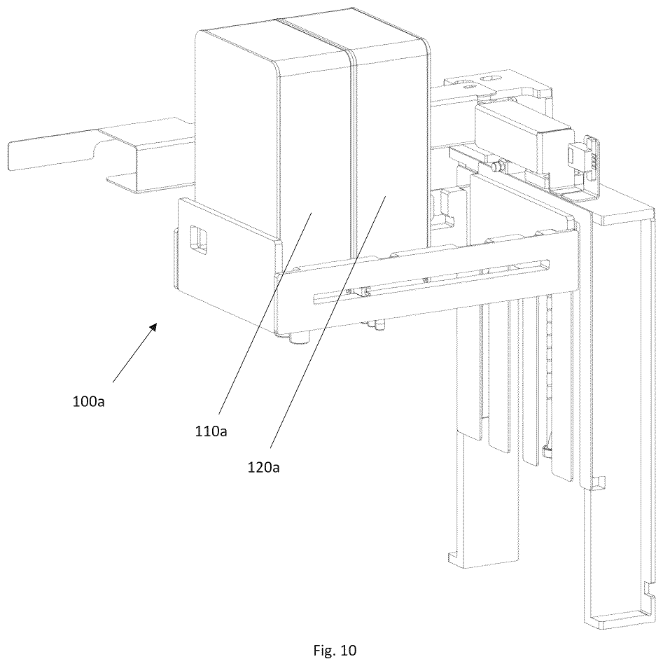

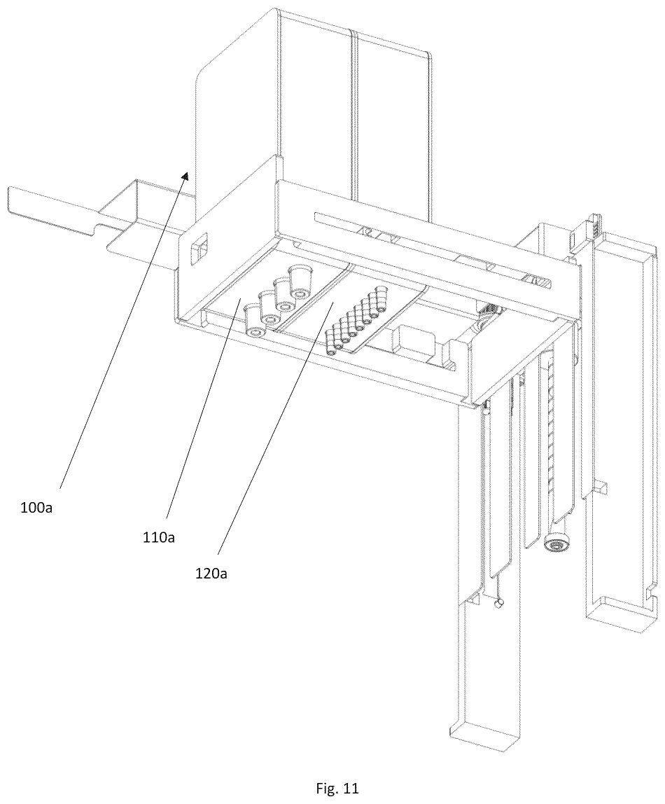

In another embodiment of the present disclosure, as shown in FIG. 8 to 11, automated system 2 is provided. The basic structures of automated systems 1 and 2 are almost the same, which will not be described here repeatedly. The major difference between automated system 1 and 2 is the switchable module. The switchable module of automated system 1 is a rotary switchable module 100, which comprises magnetic rotary mixers 110/120 and can be switched by horizontally rotation. The switchable module of automated system 2 is a linear switchable module 100a, which comprises magnetic rotary mixers 110a/120a and can be switched by horizontally movement. Rotary switchable module 100 may switch the number of channels by horizontally rotate the magnetic rotary mixers 110/120. The linear switchable module 100a may switch the number of channels by linearly move the magnetic rotary mixers 110a/120a. As the result, both of rotary switchable module 100 and linear switchable module 100a can apply with different plate by switching the magnetic rotary mixers.

With the linear switchable module 100a, the width of the auto stage 200 may be reduced and the total size of the automated system 2 may also be reduced. Specifically, the magnetic rotary mixers 110a/120a will perform the predetermined assay with horizontal movement rather than the rotary switch thereof.

On the other hand, the assays in the examples below may be performed by both automated systems 1 or 2, and the procedures are all performed in one operation.

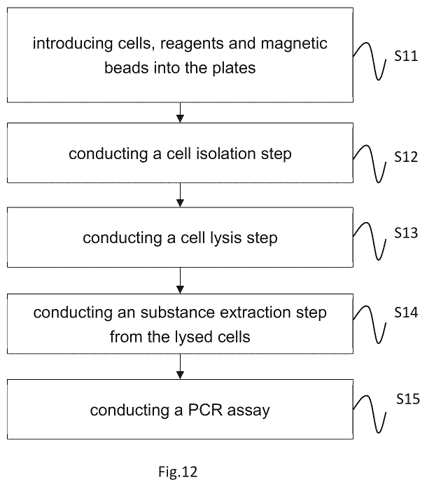

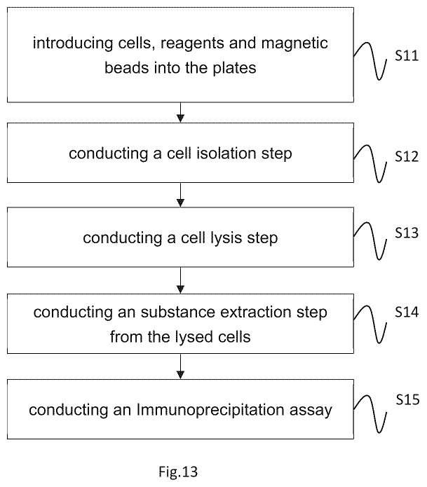

Herein after, the particularly embodiment of performing assays by automated system 1 will be described. Please refer to FIGS. 12 and 13, in one embodiment, a method for cell isolation and extracting substances by using the above mentioned automated system 1, comprising: (S11) introducing cells, reagents and magnetic beads into the plates; (S12) conducting a cell isolation step; (S13) conducting a cell lysis step; and (S14) conducting a substance extraction step from the lysed cells; wherein the cell isolation step S12, cell lysis step S13 and/or substance extraction step S14 are completed on the automated system 1 at once. The extractable substance herein may be protein, nucleic acid or any substance that can combine with the magnetic beads. In one another embodiment, the method may further comprises (S15) amplifying nucleic acid step to follow or replace step S14. Step S15 may comprise PCR, qPCR, RT-PCR, RT-LAMP etc. In still another embodiment, the step S15 may be following assays for other extracted substance, such as Immunoprecipitation etc.

In one embodiment, a method for sample isolation and/or extraction by using the above mentioned automated system 1, comprising: introducing samples, reagents and magnetic beads into the plates; conducting a sample isolation and/or extraction steps; wherein the sample isolation and/or extraction steps are completed on the automated system at once. The isolation step may not limit to cell isolation, any biomaterial may also be performed in the automated system 1.

Within the above-mentioned methods, the steps thereof are performed by a pre-determined program preloaded in the controller 300 of the automated system 1. The rotary switchable module 100 may switch the magnetic rotary mixers 110/120 according to the program preloaded in the controller 300 having different number of channels and proceed the movement to blend and/or move the sample.

In one embodiment, the isolated/extracted samples (cell, substance or others) may be transferred to the following detections or experiments, such as fluorescent test, culture test, cytotoxicity test, etc. Those following detections or experiments can be also integrated in the automated system 1.

### EXAMPLES

### Example 1—Automatic Magnetic Bead Cell Isolation

To verify the efficacy of TANPURE CD3 Magnetic Beads in isolating CD3+ cells from PBMCs by Automated system 1.

### Materials

### Instrument

Automated system 1

### Magnetic Beads

TANPURE CD3 Magnetic Beads

### Sample

Normal human blood (collected in an EDTA tube)

### Reagents

PBS, 10× Concentrate (BioLegend, 926201, Lot. B357892)
Histopaque-1077 (Sigma, 10771-100 ML, Lot. RNBL2641)
Leocosep tube, 50 mL (Greiner, 227290, Lot. E22033TQ)
RPMI-1640 medium, HEPES (Gibco, 22400-089 500 ml, Lot. 2458423)
10% in-activated FBS (prepare from Gibco, 10437-028 500 ml, Lot. 2199672RP), 1% penicillin/streptomycin (prepare from CORNING, 30-002-CI, Lot 30002376)
2 mM L-glutamine (prepare from CORNING, 25-005-CI, Lot 13122006)
Incubation/Binding/Wash buffer: 1×PBS with 0.5% BSA (Sigma, A7030-50G, Lot.
SLCF3531) and 2 mM disodium EDTA (Scharlau, 6381-92-6)
Releasing buffer: Accutase cell detachment solution (Innovative Cell Technologies, AT104, Lot. 2V2614A)
FACS buffer: 1×PBS with 2% BSA
CD3 antibody (BioLegend, 317347, B362644)
Alexa Fluor®488 anti-human CD3 (AF488-CD3, BioLegend, 300320, Lot. B363393)
Brilliant Violet 711™ anti-human CD4 Antibody (BV711-CD4, BioLegend, 317440, Lot. B346868)
Pacific Blue™ anti-human CD8 Antibody (PB-CD8, BioLegend, 344718, Lot. B358719)
7-AAD Viability Staining Solution (BioLegend, 420404, Lot. B368832)

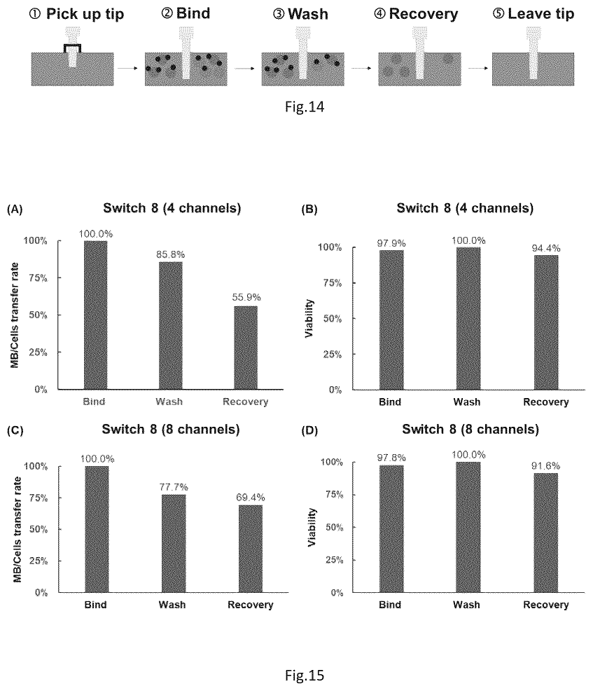

The cell isolation process can be divided into 3 steps: (1) incubate the cells and magnetic beads on ice, (2) wash away non-specific binding, and (3) recover the specific-targeted cells from magnetic beads. In this preliminary test, the 3 isolation steps are combined and processed them in automated system 1. The spin tips 103 were first picked up on the outside of magnetic rod 111, making the magnetic beads mobile. Subsequently, binding, washing and recovery by whirl stirring mixing technology were the three key steps of cell isolation, and the purpose of each step is as follows. The Flowchart of the automatic cell isolation process in automated system 1 is illustrated in FIG. 14. There are 5 main cell isolation steps in automated system 1, which are picking up spin tips, binding, washing, recovery, and leaving spin tips. Dark blue cube indicates wells of the Plate 202. Light blue indicates spin tips. Black circles represent magnetic beads. Yellow circles represent target cells. Green circles represent non-target cells.

The binding step is to label target cells with specific magnetic beads. The washing step is to remove non-specifically bound cells from magnetic beads. The recovery step is to harvest target cells from magnetic beads. During the process of cell isolation, the binding capacity of magnetic beads, viability and purity of isolated cells were confirmed in subsequent cell counting and flow cytometry.

### Procedure

### Sample Preparation: Isolation and Culture of PBMCs

Pre-warm the Leucosep tube containing 15 mL Histopaque-1077 to RT.
1. Transfer blood to a 50 mL tube and dilute 1:1 with 1×PBS.
2. Add the diluted blood to a Leucosep tube and centrifuge at 1000×g for 10 minutes without brake.
3. Remove the plasma fraction and harvest the enriched cell fraction into a 15 mL tube.
4. Add 1×PBS to 10 mL and centrifuge at 500×g, 4° C., for 10 minutes with brake.
5. Discard the supernatant and transfer the cells to a 1.5 mL tube with 1 mL of 1×PBS.
6. Centrifuge the cells at 500×g, 4° C., for 10 minutes with slow brake.
7. Discard the supernatant, resuspend the cell pellet with 1 mL of 1×PBS, and then count the cells with a Countess 3 Automated Cell Counter.
8. Seed PBMCs at a density of 2×10⁶ cells/ml in culture medium. Cells were cultured in a humidified incubator at 37° C. with 5% CO₂.

### Automatic CD3+ T Cell Isolation Using TANPURE CD3 Magnetic Beads on Automated System 1

### Working Bead Preparation

1. Place 40 μL (0.4 mg) of TANPURE CD3 Magnetic Beads into a 1.5 mL tube.
2. Add 500 μL of Incubation/Binding/Wash buffer to wash the beads and vortex gently to mix.
3. Collect the beads with the magnetic separator and discard the supernatant.
4. Repeat the washing steps for 3 times.
5. Resuspend beads in 40 μL of Incubation/Binding/Wash buffer.

### Working Cell Preparation

1. Transfer cultured PBMCs into 15 mL tube and centrifuge at 500×g for 5 minutes.
2. Discard the supernatant and resuspend the cell pellet with 5 mL of 1×PBS.
3. Centrifuge the cells at 500×g for 5 minutes.
4. Discard the supernatant, resuspend the cell pellet with 1 mL of 1×PBS, and then count the cells with a Countess 3 Automated Cell Counter.
5. Centrifuge the cells at 500×g for 5 minutes.
6. Discard the supernatant and resuspend the cell pellet with an appropriate volume of Incubation/Binding/Wash buffer so that there are 2×10⁶ cells in 180 μL.

### Automatic Isolation of CD3+ T Cells

1. Add 20 μL of working beads and 180 μL working cells into plate 202 well 1 and mix well by pipetting.
2. Place the covered plate 202 on ice for 15 minutes.
3. Place the plate 202 in automated system 1, which had the spin tips 103 set and UV sterilized.
4. Start the program as described in Tables 1 and 2.
5. Carefully remove the plate 202 while the program is finished.
6. Transfer the solution from plate well 5 to a 1.5 mL tube.

TABLE 1
Program setting of automated system 1 for isolating CD3+ cells.

| Well | Name | Volume (μL) | Action |
|---|---|---|---|
| 1/7 | IB | 200 | For. |
| 2/8 | — | — | For. |
| 3/9 | WB1 | 1000 | For. |
| 4/10 | WB2 | 1000 | For. |
| 5/11 | EB | 500 | For. |
| 6/12 | — | — | For. |

TABLE 2
Program setting of automated system 1 for isolating CD3+ cells.

| | | | Mix- | Mix- | Collec- | | |
|---|---|---|---|---|---|---|---|
| | | | ing | ing | tion | Vapor | |
| | | Temp. | (min- | speed | (min- | (min- | |
| Step | Well | (° C.) | ute) | (rpm) | ute) | ute) | Pause |
| 1 | 1/7 | Off | 0.1 | 500 | 2 | 0 | Off |
| 2 | 3/9 | — | 0.1 | 500 | 2 | 0 | Off |
| 3 | 4/10 | — | 0.1 | 500 | 2 | 0 | Off |
| 4 | 5/11 | — | 0.1 | 500 | 0 | 0 | Off |
| 5 | 2/8 | — | 0.1 | 500 | 0 | 0 | Off |

Recovery of CD3+ T cells

1. Centrifuge the solution from the previous step at 400×g for 5 minutes at 4° C.
2. Place the 1.5 mL tube on the magnetic separator and discard the supernatant.
3. Add 300 μL of Releasing buffer, mix well and incubate at 25° C. for 5 minutes.
4. Add PBS to 1 mL to neutralize the reaction.
5. Place the 1.5 mL tube on the magnetic separator and transfer the supernatant to another clean 1.5 mL tube.
6. Centrifuge the 1.5 mL tube at 400×g for 5 minutes at 4° C. and discard the supernatant.
7. Add 300 μL of 1×PBS to resuspend the cells.
8. Manually count cells with 0.4% Trypan blue and culture the cells in culture medium at a density of 1×10⁵ cells/mL.
9. Cells are cultured in a humidified incubator at 37° C. with 5% CO₂. Cell number, viability and relative cell proliferation (fold change) were calculated and analyzed on Day 0 and Day 3.

### Flow Cytometry

1. Adjust the recovered cell density to 1×10⁶ cells/ml. Aliquot 100 μL (1×10⁵ cells) into each 1.5 ml tube.
2. Add 1 μL of each flow cytometry antibody, mix well, and incubate on ice for 30 minutes in the dark.
3. Add 1 mL of 1×PBS to wash the cells, and centrifuge at 500×g for 5 minutes at 4° C.
4. Remove supernatant and resuspend cells in 1 mL of FACS buffer.
5. Mash cells through the cell strainer on the cap of flow tube.
6. Cells were analyzed by SONY SH800S Cell Sorter.

### Results

PBMCs were incubated with TANPURE CD3 Magnetic Beads from which CD3+ T cells were automatically isolated by automated system 1. During the binding, washing and recovery steps of the cell isolation, the number of non-specific and specific binding cells (CD3+ T cells) was counted, and the purity of specific binding cells was determined. The results of CD3+ T cells isolated by the 4 channels of Automated system 1 showed that 74% of input cells were collected (data not shown), 14.2% of non-specific bound cells were washed away, and 44.1% of cells were recovered after treatment with Releasing buffer (FIG. 15 (A)). Moreover, cell viability was over 94% at each step (binding, washing and recovery), indicating that these automatic cell isolation steps did not damage cells (FIG. 15 (B)). On the other hand, the results of CD3+ cells isolated by the 8 channels of Automated system 1 showed that 47% of input cells were collected (data not shown), 22.3% of non-specific binding cells were washed away, and 30.6% of cells were recovered after treatment with Releasing buffer (FIG. 15 (C)). And over 91% cell viability at each step (FIG. 15 (D)).

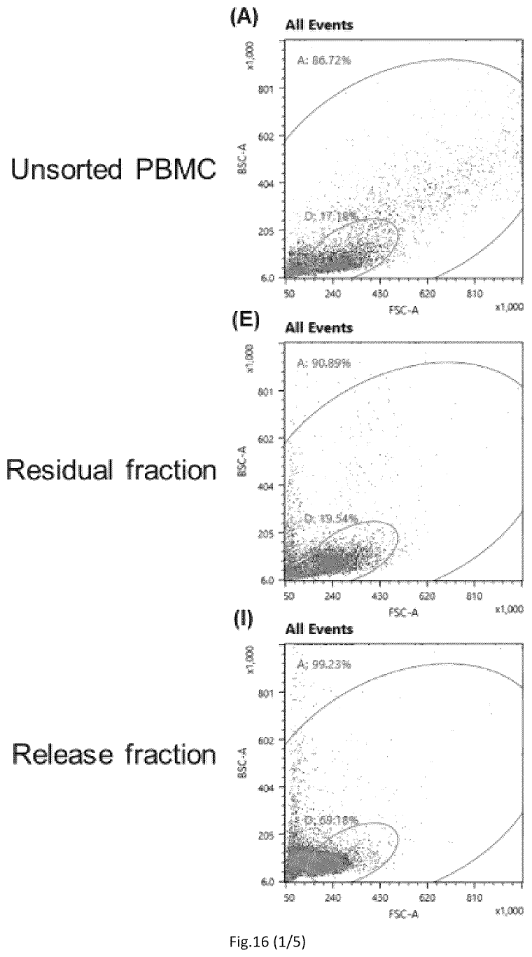

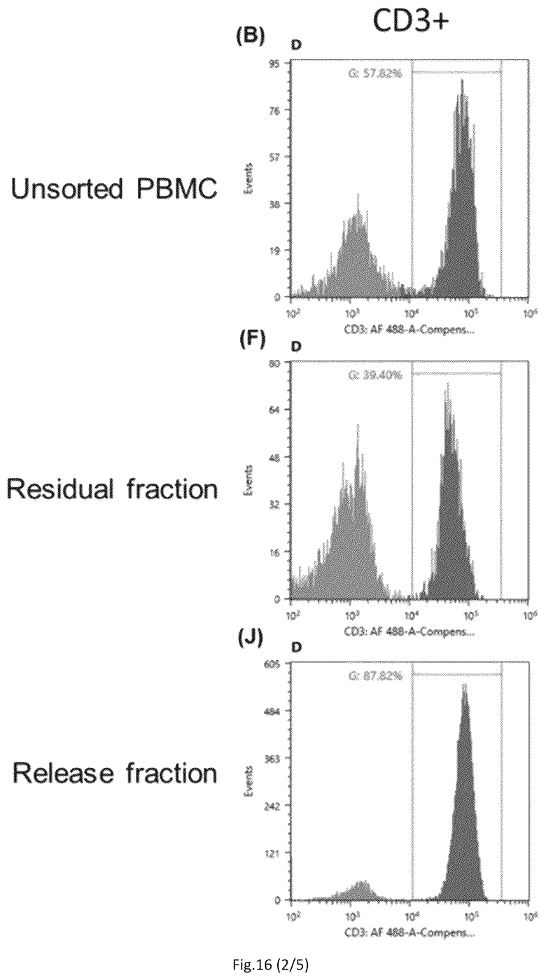

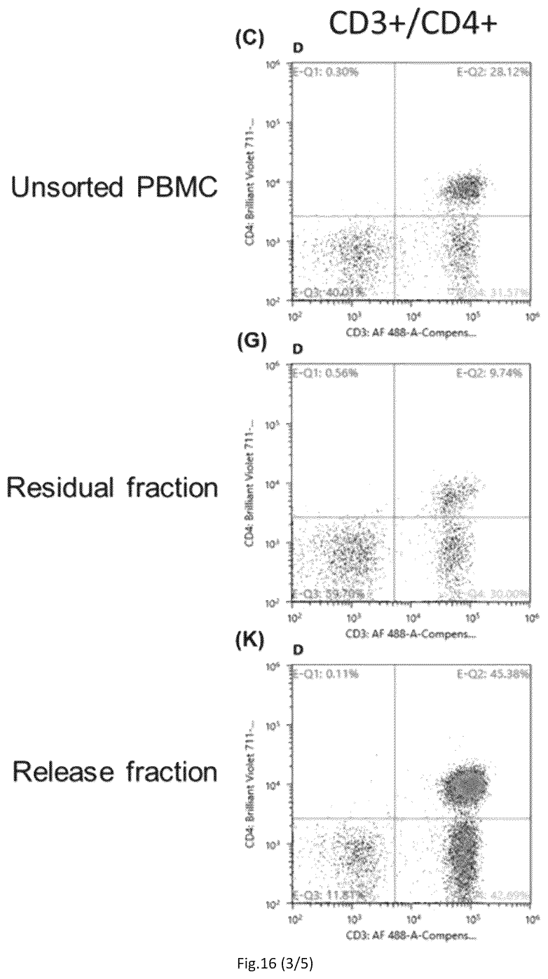

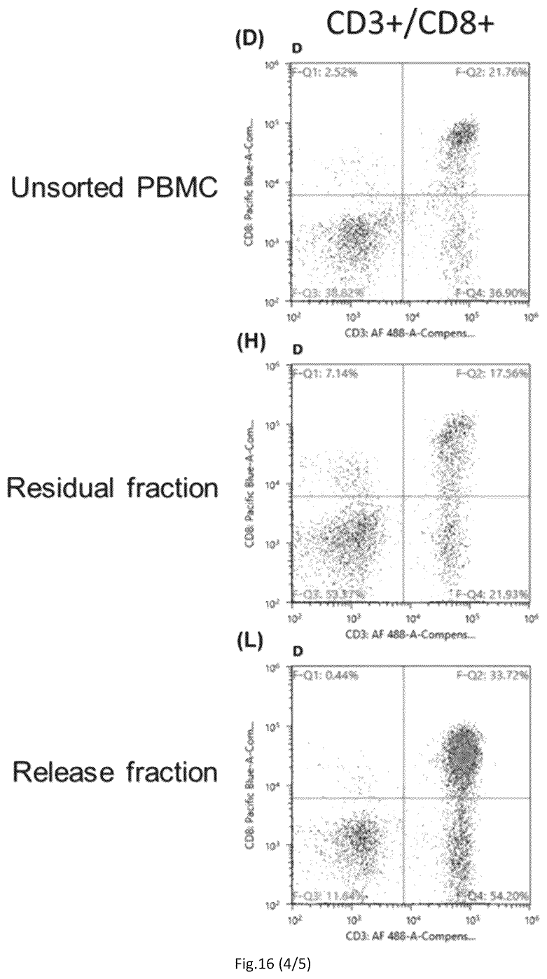

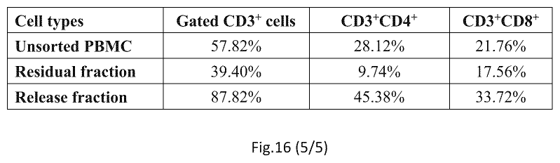

The original PBMCs were set as the unsorted control. After automatic isolation by automated system 1, cells were separated into two subpopulations of cells, cells in the residual fraction and cells in the release fraction. Cells in the residual fraction were not captured by the magnetic beads, while cells in the release fraction (enriched CD3+ T cells) were specifically captured by the magnetic beads and released after enzymatic recovery. Next, these unsorted control PBMCs, cells in the residual fraction, and cells in the release fraction were stained with AF488-CD3, BV711-CD4 and PB-CD8 antibodies and further analyzed by flow cytometry. According to the results, the percentage of CD3+ T cells in unsorted control PBMCs was 57.82%, among which were 28.12% of CD3+ CD4+ and 21.76% of CD3+CD8+ cells (FIGS. 16 (B), (C) and (D)). After automatic isolation by the 4 channels of automated system 1, the percentage of CD3+ T cells in the residual fraction was 39.40%, in which the percentages of CD3+CD4+ and CD3+CD8+ T subpopulations decreased to 9.74% and 17.56%, respectively (FIGS. 16 (F), (G) and (H)). Importantly, the percentage of CD3+ cells in the release fraction was successfully enriched to 87.82% (FIG. 16 (J)). The percentages of CD3+CD4+ and CD3+CD8+ T cell subpopulations in the release fraction were also enriched to 45.38% and 33.72%, respectively (FIGS. 16 (K) and (L)).

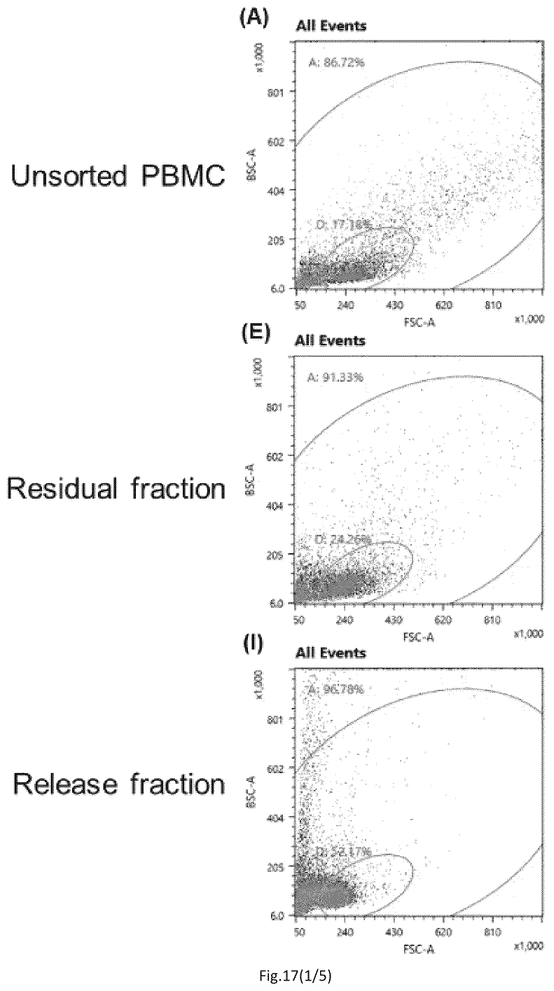

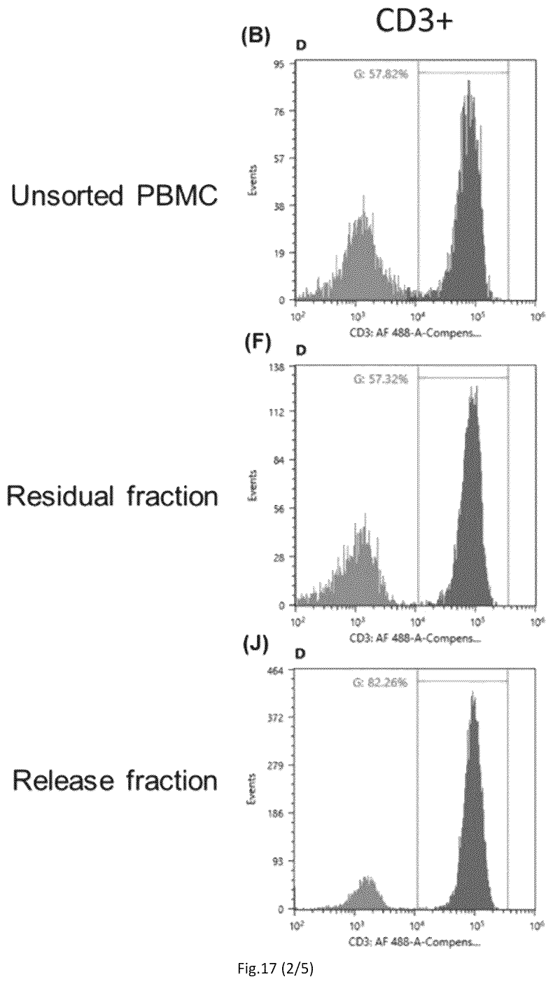

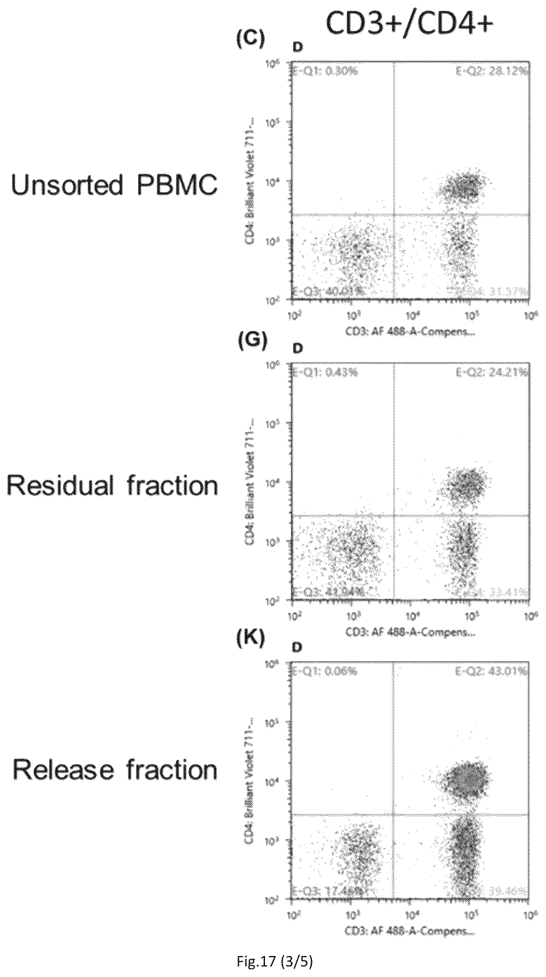

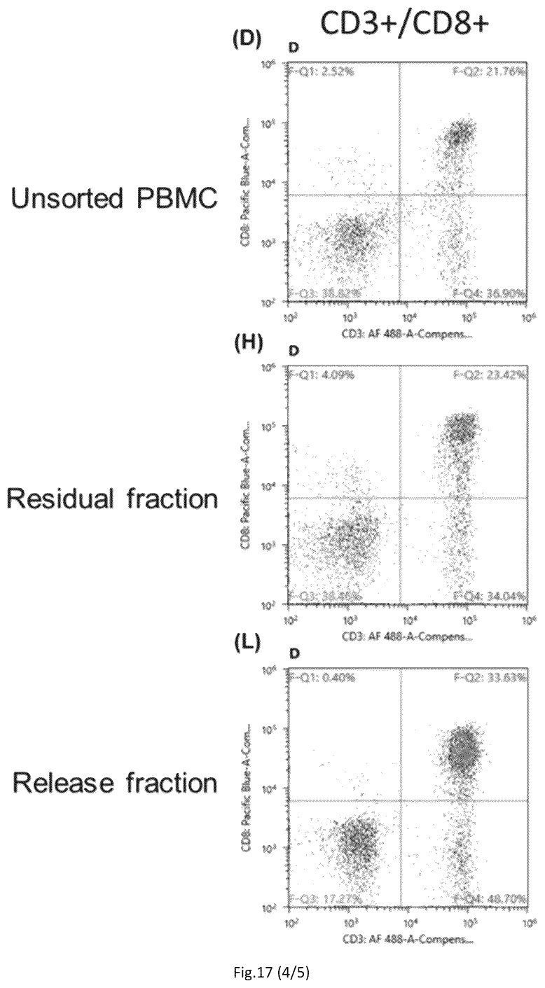

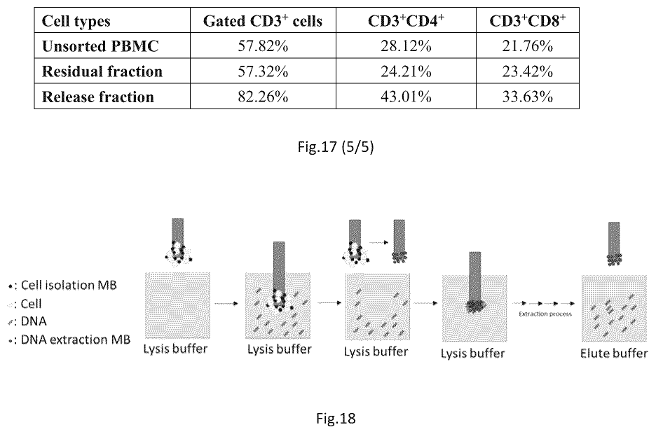

Likewise, the performance of automatic cell isolation was also demonstrated in the 8 channels of automated system 1. The percentage of CD3+ T cells in the residual cell fraction was 57.32%, in which the percentages of CD3+CD4+ and CD3+CD8+ T cells were decreased to 24.21% and 23.42%, respectively (FIGS. 17 (F), (G) and (H)). Importantly, the CD3+ cells in the release fraction were enriched to 82.26% (FIG. 17 (J)). The percentages of CD3+CD4+ and CD3+CD8+ cells in the release fraction were also enriched to 43.01% and 33.63%, respectively (FIGS. 17 (K) and (L)).

These automatically isolated CD3+ cells were further cultured to confirm whether the isolated cells could propagate in vitro. These CD3+ cells were cultured for three days and then counted. On Day 3, the viability of cultured cells from the 4 channels of automated system 1 was 97.1% (Table 3) and that from the 8 channels was 98.3% (Table 4). Relative cell proliferation of CD3+ cells isolated by the 4 channels of automated system 1 was 1.2-fold (Table 3) and 2.3-fold by the 8 channels (Table 4). In summary, these results indicated that the isolated cells after automatic isolation remained viable and could be used for further in vitro culture.

TABLE 3

| Automated system 1 (4 | | |
|---|---|---|
| channels | Day 0 | Day 3 |
| Relative cell proliferation | 1.0 | 1.2 |
| (Fold change) | | |
| Viability (%) | 94.4% | 97.1% |

TABLE 4

| Automated system 1 (8 | | |
|---|---|---|
| channels) | Day 0 | Day 3 |
| Relative cell proliferation | 1.0 | 2.3 |
| Fold change) | | |
| Viability (%) | 91.6% | 98.3% |

Here, we introduced a novel and efficient pipeline, compose of TANPURE Magnetic Beads and automated system 1 instruments with whirl stirring mixing technology, for the automatic cell isolation. The key results were that the purity of CD3+ cells isolated by the 4 channels of automated system 1 was enriched from 57.82% to 87.82%, and that of 8 channels was enriched to 82.26%. Moreover, cell viability exceeded 60% at each step of cell isolation, and the isolated CD3+ cells were propagated in vitro for three days with cell viability exceeding 95%. Yet the recovery rates of CD3+ T cells isolated by the 4 and 8 channels of automated system 1 were 44.1% and 30.6%, respectively. In conclusion, automated isolation and enrichment of CD3+ T cells from human PBMCs using TANPURE CD3 Magnetic beads and automated system 1 is feasible and highly recommended. In the future, we will also optimize the cell-to-bead ratio, buffer volume, operating program, and cell recovery conditions to improve cell isolation performance.

### Example 2—Magnetic Bead-Cell Complex for Nucleic Acid Extraction

### Materials

### Instrument

Automated system 1
Nanodrop 2000 (Thermo)

### Reagent

TABLE 5
Magnetic bead Nucleic Acid Extraction kit, 61E

| Well | Buffer | Volume (μL) |
|---|---|---|
| 1/7 | Lysis Buffer | 800 |
| 2/8 | N/A | 300 |
| 3/9 | Washing Buffer 1 | 800 |
| 4/10 | Magnetic Beads + | 50 + 750 |
| | Washing Buffer 2 | |
| 5/11 | Washing Buffer 3 | 800 |
| 6/12 | Elution Buffer | 100 |

TABLE 6
Magnetic bead Nucleic Acid Extraction kit, 612

| Well | Buffer | Volume (μL) |
|---|---|---|
| 1/7 | Lysis Buffer | 800 |
| 2/8 | N/A | 300 |
| 3/9 | Washing Buffer 1 | 800 |
| 4/10 | Magnetic Beads + | 50 + 750 |
| | Washing Buffer 2 | |
| 5/11 | Washing Buffer 3 | 800 |
| 6/12 | Elution Buffer | 130 |

### Sample

NHS-Magnetic bead (NHS-MB): Magnetic beads containing NHS functional group (NHS: N-hydroxysuccinimide
CD3-Magnetic bead (CD3-MB): Magnetic beads conjugated with CD3 antibody Pure blood DNA (60 ng/μL)
Jurkat cells

### Prepare Samples

Jurkat cell—NHS-MB: Jurkat cells mixed together with NHS-MB
Jurkat cell—CD3-MB: Jurkat cells mixed together with CD3-MB
Jurkat cell sample: a total of 1×10⁶ cells
Samples were loaded into Plate 202 of the 612 and/or 61E kit.
Extract cellular nucleic acids on automated system 1 using 612 and/or 61E kits (FIG. 18). The extraction program for 612 and 61E are listed as follows.
Measure the concentration, purity and quality of extracted nucleic acids by Nanodrop.

FIG. 18 shows flowchart of nucleic acid extraction from MB-cells.

Use lysis buffer to break up the cells and release DNA. Nucleic acid extraction is then performed using DNA extraction MB. (Black circle: cell isolation MB; blue circle: cell; DNA shape: DNA; green circle: DNA extraction MB)

TABLE 7
612 kit (modified from the original configuration)

| Well | #1 | #2 | #3 | #4 | #5 | | #6 |
|---|---|---|---|---|---|---|---|
| Content | Lysis | Sample | Washing | Magnetic | Washing | Washing | Elution |
| | Buffer | | Buffer 1 | Beads | Buffer 2 | Buffer 3 | Buffer |
| Volume(μL) | 800 | 300 | 800 | 50 | 750 | 800 | 130 |

TABLE 8
61E kit (modified from the original configuration)

| Well | #1 | #2 | #3 | #4 | #5 | | #6 |
|---|---|---|---|---|---|---|---|
| Content | Lysis | Sample | Washing | Washing | Magnetic | Washing | Elution |
| | Buffer | | Buffer 1 | Buffer 2 | Beads | Buffer 3 | Buffer |
| Volume(μL) | 500 | 300 | 800 | 1200 | 35 | 1165 | 100 |

B501 (5S) and B251 (10S) are lysis buffers.

### Program:

TABLE 9
612 program

| Program name | | 612 -AACC | | | | | |
|---|---|---|---|---|---|---|---|
| Step | Well | Temp | Mix_time | Mix_speed | Collect_time | Vapor_time | Pause |
| 1 | 4 | | 0.5 | 2500 | 0.5 | 0 | Off |
| 2 | 3 | | 0.5 | 2500 | 0.5 | 0 | Off |
| 3 | 1 | 55 | 10 | 3000 | 0.5 | 0 | Of |
| 4 | 3 | | 2 | 2500 | 0.5 | 0 | Off |
| 5 | 4 | | 1 | 2500 | 0.5 | 0 | Off |
| 6 | 5 | | 1 | 2500 | 0.5 | 0 | Of |
| 7 | 6 | 45 | 6 | 2500 | 0.5 | 0 | Off |
| 8 | 4 | | 0.5 | 2500 | 0 | 0 | Off |

TABLE 10
61E program

| Program name | | 61E - AACC | | | | | |
|---|---|---|---|---|---|---|---|
| Step | Well | Temp | Mix_time | Mix_speed | Collect_time | Vapor_time | Pause |
| 1 | 5 | | 0.5 | 3000 | 0.5 | 0 | Off |
| 2 | 1 | 70 | 10 | 3000 | 1 | 0 | Oft |
| 3 | 3 | | 2 | 3000 | 0.5 | 0 | Off |
| 4 | 4 | | 3 | 3000 | 0.5 | 0 | Off |
| 5 | 5 | | 3 | 3000 | 0.5 | 5 | Off |
| 6 | 6 | 60 | 10 | 3000 | 1.5 | 0 | Off |
| 7 | 5 | | 0.2 | 3000 | 0 | 0 | Off |

### Results

Jurkat cell-NHS-MB and Jurkat cell were used as samples. First, lyse Jurkat cell-NHS-MB and then remove magnetic beads. Subsequently, DNA was extracted using Magnetic bead 612 and 61E kits. 61E kit exhibited better nucleic acid performance than 612 and was therefore the extraction kit used in the following experiments (Table 11). And it is considered 61E to be the appropriate extraction kit for subsequent optimization of this project. Based on the results, when using Jurkat cell-NHS-MB for nucleic acid extraction by 612 and 61E kits, the quantity and quality of extracted DNA was lower compared to using Jurkat cell alone. This suggested that removal of magnetic beads by lysis was not sufficient or magnetic beads had nucleic acid affinity (Table 11).

TABLE 11
Nucleic acid extraction performance of Magnetic bead
612 and 61E kit on MB-cell complex and cells.

| | | | DNA | Nucleic | | |
|---|---|---|---|---|---|---|
| Extraction | | | Yield | acid | 260/ | 260/ |
| kit | Sample | Cell No. | (μg) | (ng/μL) | 280 nm | 230 nm |
| 612 | Jurkat | 1 × 10⁶ | 1.23 | 11.80 | 1.95 | 0.09 |
| | cells-NHS- | | | | | |
| | MB | | | | | |
| | Jurkat | | 8.48 | 81.50 | 1.85 | 0.45 |
| | cells | | | | | |
| 61E | Jurkat | | 0.22 | 2.80 | 1.64 | 0.64 |
| | cells-NHS- | | | | | |
| | MB | | | | | |
| | Jurkat | | 11.95 | 149.40 | 1.98 | 2.22 |
| | cells | | | | | |

*260/280 nm, 260/230 nm ratio are the ratio of absorption thereof

The low concentration of DNA extracted by magnetic beads from MB-cell complex. Pure DNA only and pure DNA mix together with NHS-MB or BSA-NHS-MB were used as samples for nucleic acid extraction. Blocking of NHS-MB with BSA showed high DNA yield compared to NHS-MB (Table 12).

TABLE 12
Nanodrop measurement of DNA extracted from DNA-MB
complexes and DNA alone and determination of the
function of BSA on DNA-MB complexes

| | | DNA | Nucleic | | |
|---|---|---|---|---|---|
| Extraction | | Yield | acid | 260/280 | 260/230 |
| kit | Sample | (μg) | (ng/μL) | nm | nm |
| 61E | DNA + NHS-MB | 7.7 | 96.6 | 1.81 | 2.11 |
| | DNA + BSA-NHS- | 12.0 | 150.1 | 1.8 | 2.18 |
| | MB | | | | |
| | DNA | 11.6 | 144.8 | 1.84 | 2.17 |

Note:
18 μg DNA was loaded in each individual experiment.

On the other hand, Jurkat cells-CD3-MB and Jurkat cell are used as samples. Similar to Jurkat cell-NHS-MB, DNA extracted from Jurkat cells-CD3-MB was lower in quantity and quality compared to Jurkat cell alone (Table 13). Furthermore, we demonstrated that CD3-MB also had nucleic acid affinity as 5 μg of nucleic acids were lost compared to DNA alone (Table 14). Therefore, it is believed that magnetic beads have nucleic acid affinity, and the amount of extracted DNA product can be increased by blocking magnetic beads

TABLE 13
Nanodrop measurement of DNA extracted from Jurkat
cell-CD3-MB complexes and Jurkat cell alone.

| | | | DNA | Nucleic | | |
|---|---|---|---|---|---|---|
| Extraction | | | Yield | acid | 260/ | 260/ |
| kit | Sample | Cell No. | (μg) | (ng/μL) | 280 nm | 230 nm |
| 61E | Jurkat cells- | 1.7 × 10⁶ | 1.2 | 15.6 | 1.60 | 0.54 |
| | CD3-MB | | | | | |
| | Jurkat cells | 1 × 10⁶ | 8.5 | 105.7 | 1.99 | 1.95 |

TABLE 14
Nanodrop measurement of DNA extracted from DNA-MB and
DNA-CD3-MB complexes and DNA alone

| | | Nucleic | | | Loss per |
|---|---|---|---|---|---|
| Extraction | | acid | | | reaction |
| kit | Sample | (ng/μL) | 260/280 | 260/230 | (0.1 mg) |
| 61E | DNA + NHS- | 24.5 | 1.86 | 1.82 | 12 μg |
| | MB | | | | |
| | DNA + CD3- | 70.8 | 1.82 | 2.25 | 5 μg |
| | MB | | | | |
| | DNA | 100 | 1.84 | 2.37 | N/A |

Note:
0.05 mg of MB was used in this experiment. And 12 μg DNA was loaded in each individual experiment.

Overall, it is confirmed that nucleic acid extraction from magnetic bead-cell complex is absolutely feasible.

### Example 3—Magnetic Bead-Cell Isolation and Nucleic Acid Extraction

Instruments, magnetic beads, sample, reagents and procedures are the same as example 1. The isolated cells are used to perform nucleic acid extraction following the procedure as example 2, and the procedures are all performed in the automated system 2 in one operation.

To verify the efficacy of isolating CD3+ cells from PBMCs by the 4 channel magnetic rotary mixer of Automated system 2 and extraction of nucleic acid from isolating CD3+ cells by the 8 channel magnetic rotary mixer of Automated system 2.

### Materials

Beads: TANPURE ES03 magnetic beads conjugated with CD3 antibody
   Sample: Normal human blood (E230217001, EDTA tube)

### Instrument

(1) Cell isolation: 4 channel of Automated system 2
(2) Nucleic acid extraction: 8 channel of Automated system 2
(3) Nanodrop 2000 (Thermo)

### Reagent

(1) PBS, 10× Concentrate (BioLegend, 926201, Lot. B357892)
(2) RPMI-1640 medium, HEPES (Gibco, 22400-089 500 ml, Lot. 2458423)
(3) 10% in-activated FBS (prepare from Gibco, 10437-028 500 ml, Lot. 2199672RP),
(4) 1% penicillin/streptomycin (prepare from CORNING, 30-002-CI, Lot 30002376)
(5) 2 mM L-glutamine (prepare from CORNING, 25-005-CI, Lot 13122006)
(6) Incubation/Binding/Wash buffer: 1×PBS with 0.5% BSA (Sigma, A7030-50G, Lot. SLCF3531) and 2 mM disodium EDTA (Scharlau, 6381-92-6)
(7) Releasing buffer: Accutase cell detachment solution (Innovative Cell Technologies, AT104, Lot. 2V2614A)
(8) Magnetic bead CD3+ Cells Isolation kit
(9) Magnetic bead Nucleic Acid Extraction kit, 61E

### Prepare Sample Cells: Peripheral Blood Mononuclear Cell (PBMC) Isolation and Culture

(1) Pre-warm Leucosep® tubes containing 15 mL Histopaque-1077 to RT.
(2) Transfer blood into a 50 mL tube and 1:1 dilute with 1×PBS.
(3) Add the diluted blood into a Leucosep® tubes and centrifuge at 1000×g without brakes for 10 minutes.
(4) Remove the plasma fraction and harvest the enriched cell fraction into a 15 mL tube.
(5) Add 1×PBS up to 10 mL and centrifuge at 500×g, 4° C., with brakes for 10 minutes.
(6) Discard the supernatant and transfer the cells with 1 mL of 1×PBS to a 1.5 m L tube.
(7) Centrifuge the cells at 500×g, 4° C., with brakes for 10 minutes.
(8) Discard the supernatant and resuspend the cells pellet with 1 mL of 1×PBS then count the cells with Countess™ 3 Automated Cell Counter.
(9) Seed PBMCs at the density of 2×10⁶ cells/ml in culture medium. The cells are maintained in a humidified incubator at 37° C. with 5% CO₂.

### Prepare Working Cells

(1) Transfer cultured PBMCs into 15 mL microcentrifuge tube and centrifuge at 500×g for 5 minutes.
(2) Discard the supernatant and resuspend the cells pellet with 5 mL of 1×PBS.
(3) Centrifuge the cells at 500×g for 5 minutes.
(4) Discard the supernatant and resuspend the cells pellet with 1 mL of 1×PBS then count the cells with Countess™ 3 Automated Cell Counter.
(5) Centrifuge the cells at 500×g for 5 minutes.
(6) Discard the supernatant and resuspend the cells pellet with an appropriated volume of Incubating/Binding/Wash buffer which make 2×10⁶ cells in 180 μL.
   Automatic isolate CD3+ cells
(1) Add 180 μL working cells into auto plate well 1 of isolating CD3+ cells kit and mix well by pipetting.
(2)

TABLE 15
Magnetic bead isolating CD3+ cells kit

| Well | #1 | #2 | | #3 | #4 | #5 | #6 |
|---|---|---|---|---|---|---|---|
| Content | Binding | Magnetic | Washing | Washing | Washing | EB | — |
| | Buffer | Beads | Buffer 1 | Buffer 1 | Buffer 2 | | |
| Volume(μL) | 200 | 50 | 750 | 1000 | 1000 | 500 | — |

(3) Place the auto plate of isolating CD3+ cells kit with cover on ice for 15 minutes.
(4) Place the auto plate of isolating CD3+ cells kit and 61E kit into Automated system 2 which already set up spin tips and sterilized with UV light.

TABLE 16
Magnetic bead Nucleic Acid Extraction kit, 61E

| Well | #7 | #8 | #9 | #10 | #11 | #12 |
|---|---|---|---|---|---|---|
| Content | Lysis | Washing | Washing | Washing | Magnetic | Elution |
| | Buffer | Buffer 1 | Buffer 2 | Buffer 2 | Beads | Buffer |
| Volume(μL) | 500 | 800 | 1200 | 1200 | 1200 | 100 |

(6) Start the program as table 17 described.

TABLE 17
Program setting of automated system 2 for isolating
CD3+ cells and then nucleic acid extraction.

| Kit: Isolating CD3+ | | | | Model: 4 channel of | | | |
|---|---|---|---|---|---|---|---|
| cells kit | | | | Automated system 2 | | | |
| | | | Mix | Mix | Collect | Vapor | |
| Step | Well | Temp | time | speed | time | time | Pause |
| 1 | 1 | Off | 0.1 | 500 | 0 | 0 | Off |
| 2 | 2 | — | 0.1 | 500 | 2 | 0 | Off |
| 3 | 1 | — | 0.1 | 500 | 2 | 0 | Off |
| 4 | 2 | — | 0.1 | 500 | 2 | 0 | Off |
| 5 | 3 | — | 0.1 | 500 | 2 | 0 | Off |
| 6 | 4 | — | 0.1 | 500 | 2 | 0 | Off |
| 7 | 7 | 70 | 5 | 500 | 2 | 0 | Off |
| 8 | 2 | — | 0.1 | 500 | 0 | 0 | Off |
| | | | | Model: 8 channel of | | | |
| Kit: 61E kit | | | | Automated system 2 | | | |
| | | | Mix | Mix | Collect | Vapor | |
| Step | Well | Temp | time | speed | time | time | Pause |
| 9 | 10 | — | 0.5 | 3000 | 0.5 | 0 | Off |
| 10 | 7 | 70 | 10 | 3000 | 1 | 0 | Off |
| 11 | 8 | — | 2 | 3000 | 0.5 | 0 | Off |
| 12 | 9 | — | 3 | 3000 | 0.5 | 0 | Off |
| 13 | 10 | — | 3 | 3000 | 0.5 | 0 | Off |
| 14 | 11 | — | 3 | 3000 | 0.5 | 5 | Off |
| 15 | 12 | 60 | 10 | 3000 | 1.5 | 0 | Off |
| 16 | 3 | — | 0.2 | 3000 | 0 | 0 | Off |

(7) Carefully remove the auto plate when the program is finished.
(8) Transfer the Elution Buffer from auto plate well 12 to a 1.5 mL tube.
(9) Measure the concentration, purity and quality of extracted nucleic acids by Nanodrop.

### Result

CD3+ cells were isolated from PBMCs, followed by extraction of cellular nucleic acids using the Isolating CD3+ Cells Kit and 61E kit on Automated System 2. The concentration, purity, and quality of the extracted nucleic acids were measured using a Nanodrop. Table 18 presents the results of the nucleic acid extraction, confirming that the combination of isolating CD3+ cells and nucleic acid extraction in a single step using Automated System 2 is entirely feasible.

TABLE 18
Nucleic acid extraction performance by Isolating
CD3+ cells kit and 61E kit on automated system 2.

| | | | DNA | Nucleic | | |
|---|---|---|---|---|---|---|
| Extraction | | | Yield | acid | 260/ | 260/ |
| kit | Sample | Cell No. | (μg) | (ng/μL) | 280 nm | 230 nm |
| 61E | Culture | 1 × 10⁶ | 9.6 | 120.6 | 1.91 | 1.95 |
| | PBMCs-1 | | | | | |
| | Culture | 1 × 10⁶ | 9.5 | 118.5 | 1.83 | 1.99 |
| | PBMCs-2 | | | | | |
| | Culture | 1 × 10⁶ | 10.1 | 126.3 | 1.89 | 1.93 |
| | PBMCs-3 | | | | | |
| | Culture | 1 × 10⁶ | 9.3 | 116.6 | 1.89 | 1.98 |
| | PBMCs-4 | | | | | |

With the above mentioned technical features, the automated system 2 may perform cell isolation, substance isolation, substance extraction and following assays such as nucleic acid amplification or Immunoprecipitation assay in a single instrument. Possible pollution may be avoided, and cost of the instruments may be saved.

### Example 4—Magnetic Bead-Cell Isolation and Protein Immunoprecipitation

Instruments, magnetic beads, sample, reagents and procedures are the same as example 3. The isolated cells are used to perform protein immunoprecipitation following the procedure, and the procedures are all performed in the automated system 2 in one operation.

Immunoprecipitation (IP) is a small-scale affinity purification technique that isolates antigens using a specific antibody immobilized on a solid support, such as magnetic particles. It is one of the most widely used methods for isolating proteins and other biomolecules from cell or tissue lysates, enabling their subsequent detection by western blotting and other assay techniques.

To verify the efficacy of isolating CD3+ cells from Jurkat cell (the CD3+ expression cell line) by the 4 channel magnetic rotary mixer of Automated system 2 and immunoprecipitation of CD45 protein from isolating CD3+ cells by the 4 channel magnetic rotary mixer of Automated system 2.

### Materials

Beads: TANPURE ES03 magnetic beads conjugated with CD3 antibody

### Instrument

(1) Cell isolation: 4 channel magnetic rotary mixer of Automated system 2
(2) immunoprecipitation: 4 channel magnetic rotary mixer of Automated system 2
(3) Nanodrop 2000 (Thermo)
(4) BioTek Synergy HTX Multimode Reader (Agilent)

### Reagent

i. PBS, 10× Concentrate (BioLegend, 926201, Lot. B357892)
ii. RPMI-1640 medium, HEPES (Gibco, 22400-089 500 ml, Lot. 2458423)
iii. 10% in-activated FBS (prepare from Gibco, 10437-028 500 ml, Lot. 2199672RP),
iv. 1% penicillin/streptomycin (prepare from CORNING, 30-002-CI, Lot 30002376)
v. 2 mM L-glutamine (prepare from CORNING, 25-005-CI, Lot 13122006)
vi. Incubation/Binding/Wash buffer: 1×PBS with 0.5% BSA (Sigma, A7030-50G, Lot. SLCF3531) and 2 mM disodium EDTA (Scharlau, 6381-92-6)
vii. Releasing buffer: Accutase cell detachment solution (Innovative Cell Technologies, AT104, Lot. 2V2614A)
viii. Cell Lysis buffer: RIPA buffer is recommended
ix. Magnetic bead CD3+ Cells Isolation kit
x. Anti-CD45 antibody (ab214437, abcam)
xi. Magnetic bead CD45 immunoprecipitation kit
   Sample cells: Jurkat culture cells (the CD3+ expression Cell Line)

### Prepare Working Cells

(1) Transfer cultured Jurkat cells into 15 mL microcentrifuge tube and centrifuge at 500×g for 5 minutes.
(2) Discard the supernatant and resuspend the cells pellet with 5 mL of 1×PBS.
(3) Centrifuge the cells at 500×g for 5 minutes.
(4) Discard the supernatant and resuspend the cells pellet with 1 mL of 1×PBS then count the cells with Countess™ 3 Automated Cell Counter.
(5) Centrifuge the cells at 500×g for 5 minutes.
(6) Discard the supernatant and resuspend the cells pellet with an appropriated volume of Incubating/Binding/Wash buffer which make 1×10⁷ cells in 180 μL.

### Automatic Isolate CD3+ Cells

(1) Add 180 μL working cells into auto plate well 1 of isolating CD3+ cells kit and mix well by pipetting.

TABLE 19
Magnetic bead isolating CD3+ cells kit

| Well | #1 | #2 | | #3 | #4 | #5 | #6 |
|---|---|---|---|---|---|---|---|
| Content | Binding | Magnetic | Washing | Washing | Washing | Washing | EB |
| | Buffer | Beads | Buffer 1 | Buffer 1 | Buffer 2 | Buffer 3 | |
| Volume(μL) | 200 | 50 | 750 | 1000 | 1000 | 1000 | 500 |

(3) Place the auto plate of isolating CD3+ cells kit with cover on ice for 15 minutes.
(4) Place the auto plate of isolating CD3+ cells kit and immunoprecipitation kit into Automated system 2 which already set up spin tips and sterilized with UV light.

Table 20. Magnetic bead CD45 protein immunoprecipitation kit

| Well | #7 | #8 | #9 | #10 | #11 | #12 |
|---|---|---|---|---|---|---|
| Content | Lysis | Binding/ | Magnetic | Binding/ | Binding/ | Elution |
| | Buffer | Wash | Beads | Wash | Wash | Buffer |
| | | buffer 1 | | buffer 2 | buffer 3 | |
| Volume(μL) | 500 | 500 | 500 | 500 | 500 | 60 |

(6) Add 5 μg of CD45 polyclonal antibody or Rabbit IgG antibody (as negative control) into well #7.
(7) Start the program as table 21 described.

TABLE 21
Program setting of automated system 2 for isolating
CD3+ cells and then CD45 protein immunoprecipitation.

| Kit: Isolating CD3+ | | | | Model: 4 channel of | | | |
|---|---|---|---|---|---|---|---|
| cells kit | | | | Automated system 2 | | | |
| | | | Mix | Mix | Collect | Vapor | |
| Step | Well | Temp | time | speed | time | time | Pause |
| 1 | 1 | Off | 0.1 | 500 | 0 | 0 | Off |
| 2 | 2 | — | 0.1 | 500 | 2 | 0 | Off |
| 3 | 1 | — | 0.1 | 500 | 2 | 0 | Off |
| 4 | 3 | — | 0.1 | 500 | 2 | 0 | Off |
| 5 | 4 | — | 0.1 | 500 | 2 | 0 | Off |
| 6 | 5 | — | 0.1 | 500 | 2 | 0 | Off |
| 7 | 7 | — | 5 | 500 | 2 | 0 | Off |
| 8 | 2 | — | 0.1 | 500 | 0 | 0 | Off |

| Kit: Magnetic bead CD45 protein | | | | Model: 4 channel of | | | |
|---|---|---|---|---|---|---|---|
| immunoprecipitation kit | | | | Automated system 2 | | | |
| | | | Mix | Mix | Collect | Vapor | |
| Step | Well | Temp | time | speed | time | time | Pause |
| 9 | 7 | — | 0.5 | 500 | 0 | 20 | Off |
| 10 | 7 | — | 0.5 | 500 | 0 | 20 | Off |
| 11 | 7 | — | 0.5 | 500 | 0 | 20 | Off |
| 12 | 9 | — | 0.5 | 500 | 2 | 0 | Off |
| 13 | 7 | — | 30 | 500 | 4 | 0 | Off |
| 11 | 8 | — | 0.5 | 500 | 2 | 0 | Off |
| 12 | 10 | — | 0.5 | 500 | 2 | 0 | Off |
| 13 | 11 | — | 0.5 | 500 | 2 | 0 | Off |
| 15 | 12 | — | 10 | 500 | 4 | 0 | Off |
| 16 | 3 | — | 0.1 | 500 | 0 | 0 | Off |

(8) Carefully remove the auto plate when the program is finished.
(9) Transfer the Elution Buffer from auto plate well 12 to a 1.5 mL tube.
(10) Add 30 μL of 2×SDS-PAGE sample dye.
(11) Heat the samples at 100° C. for 10 min.

### Western Blot Analysis

(1) Block the PVDF membrane with 2% BSA in 1×TBST (0.02% Tween20) for 1 hour.
(2) Discard the Blocking Buffer and wash the membrane twice with 1×TBST for 10 min.
(3) Incubate the PVDF membrane with 10 mL of primary CD45 polyclonal antibody (1:1000 in 2% BSA in 1×TBST) overnight at 4° C. and mix gently.
(4) Remove the primary antibody, then wash three times for 10 minutes with 1×TBST.
(5) Incubate the PVDF membrane with 10 mL of secondary Donkey Anti-Rabbit IgG H&L antibody (1:5000) in 1×TBST and mix gently for 1 hour at RT.
(6) Wash the membrane three times for 10 minutes with 1×TBST.
(7) Immerse the PVDF membrane with the premixed chemiluminescent substrate for 5 seconds.
(8) Detect the chemiluminescent signals of the target proteins blotted onto the PVDF membrane using a chemiluminescence detector and perform image acquisition.

### Result

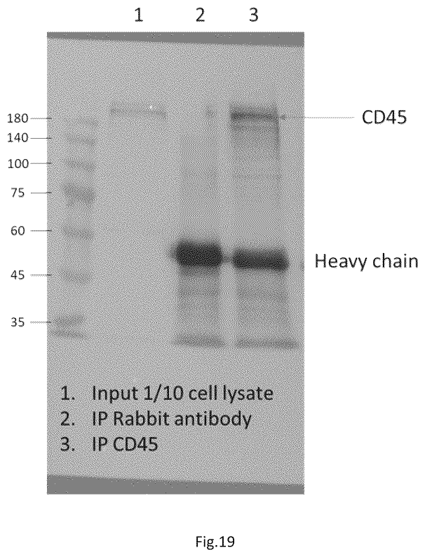

CD3+ cells were isolated from Jurkat cells (a 100% CD3+ expression cell line), and CD45 protein from these cells was then immunoprecipitated using the Isolating CD3+ Cells Kit and the immunoprecipitation kit on Automated System 2. The protein concentration of the total isolated CD3+ cells was measured using the BioTek Synergy HTX Multimode Reader from the total lysate of well #7. Table 22 compares the protein concentration of the total isolated CD3+ cells obtained through the automated process with that obtained by direct lysis of 1×10\hat{ }Jurkat cells. The results indicate that the 4-channel model of Automated System 2 achieved 93.5-98.5% of the isolation and total protein extraction efficiency. Additionally, the immunoprecipitation (IP) of CD45 protein was analyzed by western blotting. FIG. 19 shows that CD45 protein was successfully detected in the elution buffer, confirming the successful immunoprecipitation of CD45 from the total protein lysate of the isolated CD3+ cells. In conclusion, combining the isolation of CD3+ cells and the immunoprecipitation of target proteins into a single step using Automated System 2 is both feasible and effective.

TABLE 22
Isolating performance by Isolating CD3+ cells kit on automated system 2.

| | | | | | Protein | |
|---|---|---|---|---|---|---|
| | | | Protein | Protein | Yield | |
| Extraction | | Cell | Conc. | Yield | Average | Isolation |
| kit | Sample | No. | (mg/ml) | (μg) | (μg) | Efficiency |
| Isolating | Jurkat cells -1 | 1 × 10⁷ | 0.98 | 980 | 958 | 98.5% |
| CD3+ cells | Jurkat cells -2 | 1 × 10⁷ | 0.93 | 930 | | 93.5% |
| kit | Jurkat cells -3 | 1 × 10⁷ | 0.95 | 950 | | 95.5% |
| | Jurkat cells -4 | 1 × 10⁷ | 0.97 | 970 | | 97.5% |
| Manual | Jurkat cells -5 | 1 × 10⁷ | 1.00 | 1000 | 995 | 100% |
| | Jurkat cells -6 | 1 × 10⁷ | 0.99 | 990 | | |

With the above-mentioned technical features, the automated system 2 may perform cell isolation, substance isolation, substance extraction and following assays such as nucleic acid amplification or Immunoprecipitation assay in a single instrument. Possible pollution may be avoided, and cost of the instruments may be saved.

The above description is only exemplary but not limiting. Any equivalent modification or change made without departing from the spirit and scope of the present invention shall be included in the scope defined by the claims.

### CONCLUSION

With the above examples, it is obvious that the automated system disclosed in the present disclosure may save more time of the operation, and the isolation rate or extraction rate are the same or better than the traditional method. In addition, the less of operation of moving plates or loading reagents between different instruments may reduce the risk of pollution and also time-consuming.

The above description is only exemplary but not limiting. Any equivalent modification or change made without departing from the spirit and scope of the present invention shall be included in the scope defined by the claims.

What is claimed is:

1. An automated system for isolating and/or extracting substance by magnetic beads, comprising:
a switchable module, comprising a magnetic rotary mixers, the magnetic rotary mixers comprising:
a plurality of magnetic rods for generating magnetism, configured to be retractable from the switchable module;
a plurality of spin shafts for mounting spin tips, and the plurality of magnetic rods extend therein; and
a first motor for moving the plurality of magnetic rods vertically;
an auto stage, comprises:
a plate holder, which allows a plate place thereon;
a second motor for moving the plate horizontally;
a third motor for moving the switchable module vertically; and
a temperature controlling plate, disposed under the plate holder for controlling the temperature of the plate;
a controller for performing a pre-set program of movement; and
a shell housing the switchable module and the auto stage.

2. The automated system of claim 1, wherein the magnetic rotary mixers comprises a first magnetic rotary mixer and a second magnetic rotary mixer, the first magnetic rotary mixer comprises 4 or 8 channels for extending and/or retracting the magnetic rods and the second magnetic rotary mixer comprises 4 or 8 channels for extending and/or retracting the magnetic rods.

3. The automated system of claim 1, wherein the switchable module comprises rotary switchable module or linear switchable module.

4. The automated system of claim 3, wherein the rotary switchable module further comprises a fourth motor for switching the first and second magnetic rotary mixers horizontally rotation.

5. The automated system of claim 1, wherein the first to third motors are step motors.

6. The automated system of claim 1, wherein the temperature control plate is a thermoelectric sheet.

7. The automated system of claim 1, wherein the shell further comprises a HEPA.

8. The automated system of claim 7, wherein the shell further comprises an opening for the auto stage moving in and out.

9. The automated system of claim 8, wherein the opening further comprises a door.

10. The automated system of claim 9, which further comprises detection unit.

11. The automated system of claim 10, wherein the detection unit comprises fluorescent test unit, culture test unit or cytotoxicity test unit.

12. A method for sample isolation and/or extraction by using the automated system of claim 1, comprising:
introducing samples, reagents and magnetic beads into the plates; and
conducting a sample isolation and/or extraction steps;
wherein the sample isolation and/or extraction steps are completed on the automated system at once.

13. The method of claim 12, wherein the sample comprises cell or cell derivatives.

14. The method of claim 12, wherein the sample extraction step comprises extracting protein, nucleic acid or cell derivatives.

15. The method of claim 12, further comprises performing an immunoprecipitation assay.

16. The method of claim 12, which is performed by a pre-determined program in the automated system.

17. The method of claim 12, which further comprises a detection step.

18. The method of claim 17, wherein the detection step comprises fluorescent test, culture test or cytotoxicity test.
